(19) United States
(12) Patent Application Publication
(10) Pub. No.: US 2013/0022608 A1
(43) Pub. Date: Jan. 24, 2013

(54) FULL-LENGTH IgG IMMUNOGLOBULINS CAPABLE OF RECOGNIZING A HETEROSUBTYPE NEUTRALIZING EPITOPE ON THE HEMAGGLUTININ STEM REGION AND USES THEREOF

(71) Applicants: Roberto Burioni, Segrate (MI) (IT); Massimo Clementi, Milano (IT)

(72) Inventors: Roberto Burioni, Segrate (MI) (IT); Massimo Clementi, Milano (IT)

(73) Assignee: Pomona Ricerca S.r.l., Torino (IT)

(21) Appl. No.: 13/637,030

(22) PCT Filed: Mar. 25, 2011
(86) PCT No.: PCT/IB2011/051284

§ 371 Date: Sep. 25, 2012

(30) Foreign Application Priority Data
Mar. 26, 2010 (IT) TO2010A000237
Jan. 26, 2011 (IT) TO2011A000067

(51) Int. Cl. A61K 39/42; C07K 14/11; G01N 33/577; C07K 16/10; A61P 31/16

(52) U.S. Cl. 424/139.1; 530/387.9; 436/501; 530/350

(57) ABSTRACT

Monoclonal antibodies that are full-length IgG-isotype immunoglobulins and that are characterized by a high broad-range neutralizing activity against the influenza A virus and capable of recognizing a specific hemagglutinin epitope that is highly conserved among different influenza A virus subtypes are provided. Also provided are therapeutic, prophylactic and diagnostic uses of such immunoglobulins.

The present invention generally falls within the immunology field. More specifically, the invention concerns full-length immunoglobulins capable of binding and neutralizing the influenza A virus.

Antibodies directed against the influenza A virus are known in the prior art.

The International Patent Application PCT/IB2009/051068 describes monoclonal antibodies, preferably as Fab fragments, capable of binding a plurality of different influenza A virus subtypes (heterosubtypic immunity), such antibodies further being endowed with an important neutralization activity. The preferred antibody described in the above-mentioned International Patent Application is Fab 28 fragment, characterized in that it comprises a heavy chain variable region including the amino acid sequence SEQ ID NO:1 and a light chain variable region including the amino acid sequence SEQ ID NO:2. The respective encoding nucleotide sequences, that is SEQ ID NO:3 (heavy chain variable region) and SEQ ID NO:4 (light chain variable region) are also described in PCT/IB2009/051068. In the present specification, the antibody fragment Fab 28 is designated as “Fab PN-SIA28”.

In the International Patent Application PCT/IB2009/051068 the authors mention that the antibody subject of the invention may be alternatively provided as a full-length immunoglobulin as opposed to a Fab fragment. As known, human immunoglobulins are classified into five main classes: IgG, IgA, IgM, IgD, and IgE, which differ in the heavy chain type. However, in PCT/IB2009/051068, no specific immunoglobulin class is individually identified, and no suggestion regarding the heavy chain type is provided such as to allow the person of skill in the art to select a specific immunoglobulin class.

Moreover, in PCT/IB2009/051068 no experimental data regarding the neutralizing effectiveness of PN-SIA28 antibody when used as a full-length immunoglobulin are provided. Surprisingly, the present inventors have now found that a monoclonal antibody characterized by the amino acid sequences of the heavy chain variable region and the light chain variable region described in PCT/IB2009/051068, specifically provided as a full-length IgG-class immunoglobulin, shows a dramatic increase in the neutralizing activity towards the influenza A virus, both in terms of power and in terms of range of action, compared to the corresponding Fab fragment.

As known, an IgG is made of a pair of light chains (L) and a pair of heavy chains (C). Each light chain contains two immunoglobulin domains, one variable (VL) and one constant (CL) domain. The heavy chains are of the γ type and each of them contains a variable immunoglobulin domain (VH or Vγ) and three constant domains (CH1/2/3). The γ chains are in turn classifiable into four different subtypes, designated as γ1, γ2, γ3, and γ4, respectively.

The monoclonal antibody as a full-length IgG, characterized by the amino acid sequences of the heavy chain variable region and the light chain variable region described in PCT/IB2009/051068, is hereinafter designated as “IgG PN-SIA28”.

Table 1 schematically shows the results obtained by the present inventors regarding the neutralizing activity of IgG PN-SIA28, expressed as comparisons.

There is evidence of a high neutralizing activity towards different strains of the H₃N₂ subtype, which instead is lower in the case of the corresponding Fab fragment.

Furthermore, even at extremely low concentrations, the full-length IgG exhibits a very powerful neutralizing activity towards different strains of the H1N1 subtype, higher than that of the corresponding Fab fragment against the same H1N1 strains. The present inventors have also performed preliminary experiments that allowed to assess the ability of IgG PN-SIA28 of also binding recombinant hemagglutinins belonging to subtypes H2, H5, and H9. The obtained results, which are described in detail in the experimental section, allow to believe that the neutralizing activity of IgG PN-SIA28 extends to at least subtypes H2, H5, and H9, which had not been previously described when referring to the corresponding Fab fragment.

Given the extraordinary neutralizing properties of IgG PN-SIA28, the present inventors have also performed particularly complex studies and experiments (illustrated in detail hereinafter) which allowed them to identify the hemagglutinin region constituting the epitope specifically recognized by the neutralizing antibody IgG PN-SIA28. The identification of the neutralization epitope recognized by IgG PN-SIA28 is particularly useful and important, making it possible to identify further human monoclonal antibodies directed against the same epitope and endowed with extraordinary neutralizing properties substantially comparable to those of IgG PN-SIA28.

Thus, one first object of the present invention is a human monoclonal antibody in the form of a full-length IgG, specifically directed against the influenza A virus hemagglutinin anti-gen and capable of binding and neutralizing a plurality of influenza A virus subtypes including at least the H1 subtype and the H3 subtype, characterized in that the antibody recognizes a conserved epitope located in the hemagglutinin stem region, said epitope comprising the amino acid residues His25, His45, Thr315 and Asn336 of the HA1 hemagglutinin polypeptide and the amino acid residues Thr358, Met360, Ile361 or Va1361, Asp362, Gly363, Trp364, Yhr384, Thr392, Va1395, Glu400 of the HA2 hemagglutinin polypeptide, the numbering of the amino acid residues being based on the H1N1 hemagglutinin amino acid sequence available in the NCBI database under accession number EF467821.1 and designated in the sequence listing as SEQ ID NO: 5.

In a preferred embodiment, the human monoclonal antibody in the form of a full-length IgG which is the subject of the invention is capable of binding and neutralizing a plurality of influenza A virus subtypes including at least the H1 subtype, the H3 subtype and at least one among the H5, H2 and H9 subtypes. In another preferred embodiment, the human monoclonal antibody in the form of a full-length IgG which is the subject of the invention is capable of binding and neutralizing a plurality of influenza A virus subtypes including at least the H1 subtype, the H3 subtype and at least two among the H5, H2 and H9 subtypes. In a further preferred embodiment, the human monoclonal antibody in the form of a full-length IgG which is the subject of the invention is capable of binding and neutralizing a plurality of influenza A virus subtypes including at least the H1 subtype, the H3 subtype, the H5 subtype, the H2 subtype and the H9 subtype.

In a further preferred embodiment, the human monoclonal antibody in the form of a full-length IgG which is the subject of the invention is IgG PN-SIA28, characterized in that it comprises a heavy chain variable region comprising the amino acid sequence SEQ ID NO:1 and a light chain variable region comprising the amino acid sequence SEQ ID NO:2.

The studies that led to the definition of the epitope recognized by the IgG PN-SIA28 anti-body are described in detail in the experimental section that follows. Comparative studies carried out with reference to the epitope recognized by the prior art anti-influenza monoclonal antibodies C179, CR6261 and F10 are also described. Thanks to the comparative studies performed, the present inventors could prove that the above-mentioned prior art antibodies, which represent some of the most known and widespread anti-influenza antibodies, effectively recognize epitopes different from the epitope recognized by IgG PN-SIA28.

The following experimental section also illustrates the manufacture of IgG PN-SIA28 and the neutralization tests performed, with the relevant results.

The full-length IgG subject of the invention, preferably, but without limitation, the IgG PN-SIA28, is manufactured and used either in the free form or in a conjugated form with a carrier. A carrier is any molecule or chemical or biological entity designed to be conjugated to an antibody and modify the pharmacokinetic characteristics thereof, make it immunogenic or increase its immunogenicity. Non-limiting examples of carriers are proteins such as KLH (“keyhole limpet hemocyanin”), edestin, thyroglobulin, albumins such as bovine serum albumin (BSA) or human serum albumin (HSA), erythrocytes such as sheep red blood cells (SRBC), the tetanus anatoxin, the cholera anatoxin, poly-amino acids such as for instance poly(D-lysine:D-glutamic acid) and the like. In order to help the binding of the antibody to the carrier, the C-terminus or the N-terminus of the antibody can be modified, for instance by introduction of additional amino acid residues, for example one or more cysteine residues which are able to form disulphide bridges.

Due to its properties, illustrated in detail in the following experimental section with particular reference to IgG PN-SIA28, the full-length IgG subject of the invention is particularly suitable to be used in applications within the medical field, in particular for the manufacture of a medicament for the broad-spectrum prophylactic or therapeutic treatment of influenza A virus infections.

Thus, the use of the IgG of the invention, preferably, but without limitation, the IgG PN-SIA28, for the manufacture of a medicament for the prophylactic or therapeutic treatment of pathologies caused by influenza A virus infections, such as for example the influenza syndrome, falls within the scope of the invention.

The results of the neutralization tests performed and showed in the following experimental section induce to believe that the IgG PN-SIA28 is extremely effective in conferring a passive immunity against the influenza A virus in subjects to which such an IgG is administered, and that as a result it is particularly useful in the broad-spectrum prophylactic or therapeutic treatment of pathologies caused by influenza A virus infections, such as for example the influenza syndrome, particularly in a human being. Any other full-length IgG that recognizes the same hemagglutinin epitope which is recognized by IgG PN-SIA28 has neutralizing abilities substantially equal to those of IgG PN-SIA28.

Therefore, another object of the invention is a pharmaceutical composition comprising as the active ingredient an effective amount of the IgG of the invention, preferably, but without limitation, the IgG PN-SIA28, and a pharmaceutically acceptable carrier and/or diluent.

An effective amount of IgG is a quantity designed to fulfill a favorable effect in the subject to which the composition is administered, for instance to neutralize the influenza A virus by affecting a phase of its replication cycle.

In this context, the term “subject” refers to any animal host to which the composition is administered and performs its protective effect, preferably a mammal, more preferably a human being.

Non-limiting examples of pharmaceutically acceptable carriers or diluents useful in the pharmaceutical composition of the invention include stabilizers such as SPGA, carbohydrates (for example, sorbitol, mannitol, starch, saccharose, glucose, dextran), proteins such as albumin or casein, agents containing proteins such as bovine serum or non-fat milk, and buffers (for example phosphate buffer).

The IgG of the invention, preferably but without limitation the IgG PN-SIA28, may also be used advantageously as a diagnostic reagent in an in vitro method for detecting in a biological sample previously obtained from a patient (such as for example a serum, plasma, blood sample or any other suitable biological material, obtained from the patient, preferably a human being) anti-influenza A virus antibodies with a heterosubtype cross-neutralizing activity against the influenza A virus. These antibodies may be found in the biological sample obtained from the patient for instance as a result of a previous exposure to an influenza A virus, or because a monoclonal antibody of the invention had been previously administered to the patient for therapeutic or prophylactic or research purposes.

Therefore, an assay method for detecting, in a biological sample derived from a patient, the presence of anti-influenza A virus antibodies with a heterosubtype cross-neutralizing activity falls within the scope of the invention, the method comprising contacting said biological sample with the IgG of the invention, preferably but without limitation the IgG PN-SIA28, as a specific assay reagent.

The assay may be qualitative or quantitative. The detection and/or quantification of the anti-influenza A virus antibodies with a heterosubtype cross-neutralizing activity may be for example carried out by a competitive immunoassay, for instance a competitive ELISA, in which the ability of the biological sample previously obtained from the patient to displace the binding of the IgG of the present invention to hemagglutinin is assessed. The general features of the competitive immunoassays are generally known to the person of skill in the art and do not need a detailed description here.

Thus, a diagnostic kit comprising the IgG of the invention, preferably but without limitation the IgG PN-SIA28, as a specific reagent, also falls within the scope of the invention, said kit being in particular designed for the detection and/or quantification, in a biological sample previously obtained from a patient, of anti-influenza A virus antibodies with a heterosubtype cross-neutralizing activity against the influenza A virus.

Similarly, the IgG of the invention, preferably but without limitation the IgG PN-SIA28, may be used as a specific reagent in an assay method for detecting and/or quantifying, in a previously prepared immunogenic or vaccine composition, the epitope capable of evoking anti-influenza A virus antibodies with neutralizing properties identical or similar to those of the IgG of the invention, that is a heterosubtype cross-neutralizing activity against the influenza A virus, in the subject to which such a composition has been administered.

Such a method is predicted to be useful for the evaluation of any preparation that is to be used as a vaccine or immunogenic preparation, as the recognition by the monoclonal anti-body of the invention is indicative of the presence, in the immunogenic preparation and/or vaccine, of one or more epitopes capable of stimulating the production of antibody clones capable of recognizing an advantageous epitope, such as for example an epitope capable of evoking a heterosubtypic immunity against the influenza A virus such as the one identified by the present inventors and described in detail in the present patent specification.

Finally, the IgG of the invention, preferably but without limitation the IgG PN-SIA28, may be used for the preparation of mimotopes, such as for example anti-idiotype antibodies, peptides, hemagglutinin truncated or artificial forms or others, endowed with the ability of evoking IgG PN-SIA28-like antibodies. Among these, the anti-idiotype antibodies are preferred. The anti-idiotype antibodies are antibodies specifically directed against the idiotype of the broad-spectrum neutralizing antibodies used for the manufacture thereof, and thus are able to mimic the key epitopes that they recognize. The manufacture of anti-idiotype antibodies is carried out by per se known methodologies that do not need further detailed explanations here. The paper published by the same inventors (Burioni et al. (2008) PLoS ONE 3(10):e3423, related to the manufacture of an anti-idiotype antibody capable of mimicking a critical epitope of the human immunodeficiency virus (HIV) gp120 is mentioned by way of example.

Thus, also mimotopes, preferably anti-idiotype antibodies, directed against an IgG of the invention, preferably but without limitation the IgG PN-SIA28, fall within the scope of the invention.

The following experimental section is provided purely by way of illustration and not limitation of the scope of the invention as defined in the appended claims.

### EXPERIMENTAL SECTION

### 1. Preparation of IgG PN-SIA28 and Characterization of its Neutralizing Abilities

IgG PN-SIA28, which represents a preferred embodiment of the invention, was generated by using baculoviruses. Sf-9 cell line was used for the transfection experiments and the subsequent production of high-titer virus stocks, whereas High Five cell line (H5) was used for the production of the recombinant proteins. Both of them are maintained under serum-free conditions, by using Sf-900 II SFM medium and Express-Five SFM (Gibco), respectively. The cells were incubated at 27° C. and maintained in suspension at a concentration of about 10⁶ cells/ml.

### Preparation of the Recombinant Baculovirus

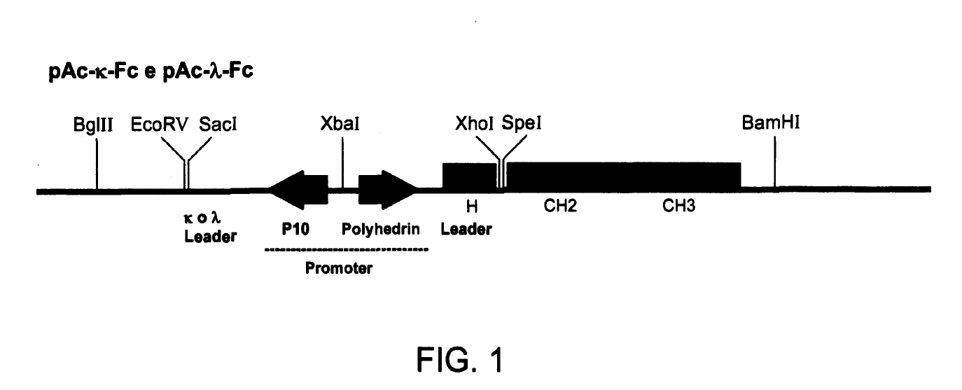

The generation of full-length IgGs starting from the Fab fragment was carried out by cloning the genes for the heavy and light chains into a transfer vector that contains regions homologous to the AcNPV baculovirus genome (genes for the promoters P10 and polyhedrin). The Baculovirus DNA (BD Biosciences Pharmingen) contains a lethal deletion, therefore it is not able to effect a productive infection once transfected into insect cells. When the baculovirus linearized genome is co-transfected into Sf-9 cells with the complementary transfer vector, the recombination between homologous regions results in the generation of a recombinant baculovirus, viable again and also able to express heterologous proteins. In particular, in order to generate the recombinant baculovirus that expresses the human full-length antibody, the transfer vector pAc-k-Fc (PROGEN) was used, which contains the Fc portion of the human IgG immunoglobulins and in which the genes for the light and heavy chains of the Fab PN-SIA28 have been cloned. The genes for the light chains were cloned into the transfer vector pAc-k-Fc by using the restriction sites Sad, EcoRV, whereas the heavy chains by using the restriction sites XhoI, SpeI. FIG. 1 shows a schematic representation of the transfer vector (Progen), wherein the cloning sites for the heavy and light chains are indicated.

The transfer vector, verified by digestion and sequencing tests, was thus co-transfected with 5 μg of Baculovirus DNA (BD), following the protocol recommended by the manufacturer (BD Bioscences). The post-transfection medium was collected after approximately 6-7 days and analyzed for the presence of human immunoglobulins in an ELISA assay. This first supernatant was designated as “step 0” (S0).

In order to obtain a pure recombinant baculovirus population, a plaque assay was performed. Briefly, 2×10⁶ Sf-9 cells were seeded into a 6-well plate and serial dilutions (from 10⁴ to 10 of the supernatant S0 were prepared. 1 ml of each dilution was added to each well and after 1 hour at 27° C. the inoculum was removed and a semi-solid medium (1% agarose solution) was added to each well. The plate was incubated at 27° C. for approximately one week, that is until the appearance of lytic plaques detectable under a microscope. Several plaques were individually transferred into wells of a 24-well plate containing 10⁵ Sf-9 cells. After about one week, each supernatant was tested in an ELISA assay for the presence of IgGs secreted into the culture medium. Moreover, it was verified, again by an ELISA assay, that the antibody maintained its binding specificity towards the antigen. In particular, the recombinant IgG PN-SIA28 antibodies secreted into the culture medium were detected by a conventional capture ELISA assay, by using a polyclonal capable of binding human Fab fragments, as the capture reagent, and a horseradish peroxidaseconjugated polyclonal capable of binding the human Fc fragment, for the detection. In order to determine the specificity of the recombinant antibody, the ELISA assay was carried out by using, as the antigen, the influenza vaccine Inflexal V (season 2007-08), formerly recognized by the monoclonal as the Fab fragment.

### Amplification of the Recombinant Baculovirus

Once obtained a pure population of recombinant baculoviruses by plaque assay, this was amplified to obtain high-titer virus stocks (approximately 10⁹ PFU/ml). Four in vitro amplification steps were performed, the first three (S1, S2, S3) in adherent Sf-9 cells, whereas the last step (S4) in suspended cells. S4 was then titrated in 96-well plates by the end-point dilution assay and the titer was calculated with the Reed-Muench formula.

### Expression of the Recombinant Antibody

In order to define the optimum expression conditions, H5 cells in suspension were infected at several Multiplicities of Infection (MOI 1, 5 and 10) and the medium was collected on different days (3, 4, 5, 6 days post-infection). The various aliquots were tested by Western Blot, both to establish the optimum infection conditions and to verify the integrity of the produced molecule. The produced recombinant immunoglobulins were detected with a horseradish peroxidase-conjugated polyclonal capable of binding the human Fc fragment. The assay was performed under denaturing and not reducing conditions (SDS 10%).

### Purification and Quantification of Recombinant PN-SIA28 IgGs

After having infected 1 L of H5 cells (MOI 5) at a concentration of 10⁶ cells/ml, the cell viability was monitored daily by Trypan Blue staining. After about 4 days (approximately 70-80% mortality), the medium was collected, centrifuged and filtered with 0.2 μm filters (Millipore). The recombinant antibody was then purified from the culture supernatant by affinity using the G protein. The antibody was eluted in 10 ml of elution buffer (0.1 M citric acid, pH 3) and concentrated by ultra-filtration through Amicon Ultra-15 (Millipore). The concentration and purity of the purified antibodies were determined by polyacrilamide gel (SDS-PAGE) and subsequent staining with Coomassie Blue, by referring to a BSA standard curve. Furthermore, the quantification of the recombinant protein was assessed by ELISA, by referring to a standard curve from human commercial IgGs.

### Micro-Neutralization Assay

MDCK cells (4×10⁴ per well) were seeded into a 96-well plate. Serial dilutions of Fab PN-SIA28 (20 μg/ml-0.078 μg/ml) and IgG PN-SIA28 (10 μg/ml-0.039 μg/ml) were incubated with 100 TCID₅₀ of each H1N1 or H₃N₂ virus. After 1 hour at 37° C., 100 μl of the virus-Fab or virus-IgG mixture at the different concentrations (for the infection control, the mixture is constituted by the virus alone) were added into each well. After 1 hour at 37° C., the 100 μl of the mixture were removed and 100 μl of MEM TPCK-Trypsin were added into each well. After about 7 hours at 37° C., the cell monolayer was fixed and permeabilized with a cold ethanol solution for 10 minutes at room temperature. The cells were then incubated for 30 minutes at 37° C. in a humidified chamber with an anti-influenza A monoclonal antibody (Argene) which was detected with a fluorescein isocyanate (FITC)-conjugated antibody. Finally, the cells were stained with the nuclear dye Hoechst 33342. The neutralizing activity of the antibodies was determined by calculating the decrease in the number of infected cells compared to the control virus, that is the virus not pre-incubated with the antibody of interest. In the experiments, a negative control was always included (virus+antibody not specific for the influenza virus), non-infected cells and a control for the toxicity of the antibodies used. The number of positive nuclei in each well was calculated by the automated GE Healthcare's IN Cell Analyzer System.

### Results

### Generation of the Full-Length Antibody Through Baculoviruses

The full-length immunoglobulins were obtained by cloning the genes for the Fab PN-SIA28 heavy and light chains into the transfer vector pAc-k-Fc (Progen) which contains the Fc portion.

After having co-transfected the Sf-9 cells with the vector pAc-k-Fc and the baculovirus linearized DNA, the inventors verified that the post-transfection culture medium contained human IgGs. Further, the IgGs secreted into the culture medium were tested for maintenance of the binding specificity. To this end, the post-transfection medium was tested by ELISA using, as the antigen, a polyclonal capable of binding human Fab fragments and the influenza vaccine Inflexal V (season 2007-08), respectively. In both cases, the monoclonal IgG PN-SIA28 was detected by a horseradish peroxidase-conjugated polyclonal capable of binding the human Fc fragment. 1% BSA was used as the negative control antigen. In parallel, the immunoenzimatic assay suitably adapted was also carried out using the Fab PN-SIA28. The ELISA results showed that the newly produced IgGs had been secreted into the culture medium after transfection, and had maintained their binding specificity, that is the ability to bind the influenza vaccine, as well as the Fab fragments derived therefrom.

The post-transfection medium was used for carrying out the plaque assay in order to obtain a pure population of recombinant baculoviruses. The supernatant from several individual plaques was tested by ELISA in order to assess the presence of IgGs secreted into the culture medium. Not all the supernatants resulted positive for the presence of immunoglobulins in the culture medium, and these were accordingly discarded.

After having obtained a high-titer virus stock (10⁸-10⁹ pfu/ml), the inventors monitored the expression of IgG PN-SIA28 by using different MOIs and different time-points. For the expression tests, the H5 cell line was used and the several medium aliquots collected on days 3, 4, 5 and 6 post-infection were tested by Western Blot, under denaturing and not reducing conditions. On the basis of the results obtained (data not shown), the inventors chose an MOI of 5 and to collect the medium on day 4 post-infection for the production of the recombinant protein in H5 cells.

### Characterization of the In Vitro Biological Activity: Micro-Neutralization Assay

Micro-neutralization assays in 96-well plates with several virus isolates belonging to the H1N1 and H3N2 subtypes were performed in order to assess the neutralizing activity of the antibodies. The experiments were carried out by using both the Fab PN-SIA28 and the IgG PN-SIA28. The results of the micro-neutralization assays are shown in the Table 1 below. The neutralizing ability of the antibodies is expressed as IC₅₀, that is the antibody concentration (expressed as μg/ml) capable of neutralizing the infection of the individual virus isolates by 50%:

TABLE 1

| | Fab 28 | IgG PN-SIA28 |
|---|---|---|
| H1N1 | | |
| A/Milan/UHSR1/2009 | 0.3 | 0.2 |
| A/WS/33 | 2.9 | 1.3 |
| A/Mal/302/54 | 0.8 | 0.64 |
| A/PR/8/34 | 2 | 1.2 |
| A/NC/20/99 | 7.0 | ND* |
| A/Swine/Parma/1/97 | 3.9 | 2 |
| H3N2 | | |
| A/Hong Kong/8/68 | 20 | 0.8 |
| A/Aichi/2/68 | 20 | 0.6 |
| A/Victoria/3/75 | >20 | 1.2 |
| A/Port Chalmers/1/73 | 13.19 | 1.8 |
| A/Wisconsin/67/2005 | >20 | ND* |

*ND: experiment not done

The assayed monoclonal resulted capable of neutralizing, both as a Fab fragment (Fab PN-SIA 28) and as a full-length immunoglobulin (IgG PN-SIA28), all the isolates tested belonging to the H1N1 subtype (reference ATCC strains and the 2009 pandemic H1N1 isolate). However, the recombinant proteins in the form of the full-length IgG show a neutralizing activity that is much higher than that of the Fab. The full-length immunoglobulin IgG PN-SIA28, in contrast with the Fab PN-SIA 28, also shows a significant neutralizing activity towards all the tested H₃N₂ subtype isolates. Therefore, on the whole, the full-length immunoglobulin IgG PN-SIA28 exhibits IC₅₀s definitely lower than those of the Fab.

### 2. Identification of the Epitope Materials and Methods

### Selection and Characterization of Mutants Capable of Escaping the Antibody Response (Anti-Body Escape Mutants) Under Fab PN-SIA 28 Selective Pressure

The experiment was performed on 90% confluent MDCK cells grown in T25 flasks in MEM supplemented with 10% FBS (fetal calf serum). Fab PN-SIA 28 and e509 anti-E2/HCV were used, the latter used as a negative control (mock control), which were diluted in 1.5 ml of MEM supplemented with TPCK trypsin (2 μg/ml) in order to obtain final antibody concentrations of 2 μg/ml, 10 μg/ml and 20 μg/ml. 100 TCID₅₀ of A/PR/8/34 (ATCC no. VR-1469™) were prepared in 1.5 ml of the same medium. The two solutions, containing Fabs and viruses, were mixed to obtain a final concentration of 1 μg/ml, 5 μg/ml and 10 μg/ml of the Fabs in a final volume of 3 ml. The mixtures were then incubated for 1 hour at 37° C. An infection positive control (virus without PN-SIA28) was also included, as well as non-infected cells. After two washes with sterile 1×PBS, 1 ml of each neutralization mixture was added to the flasks containing the MDCK cells, and an infection was carried out for 1 hour at 34° C. in the presence of 5% CO₂. After the absorbance, the medium was removed and the monolayer was washed twice with sterile PBS. Three ml of MEM supplemented with trypsin (2 μg/ml) were added to the infection positive control and to the non-infected cells. Antibodies PN-SIA28 and e509 were added to the previous-ly-treated infected cells, maintaining the concentrations used during the infection step. The cells were incubated at 34° C. with 5% CO₂ and checked regularly for 48 hours for the presence of a cytopathic effect (CPE), comparing the infection positive control with the treated infected cells. The supernatant was then collected, centrifuged (2000 rcf for 10 minutes) and stored at −80° C. All the virus stocks were titrated and used for infecting new cell preparations, increasing where possible the Fab concentration. After 10 passages, the infection positive control and negative control (mock control) cells were compared with cells infected under PN-SIA28 selective pressure. When a strong cytopathic effect was evident in the positive and negative (mock) controls, the presence or absence of CPE was assessed in the PN-SIA28-treated infected cells. All the supernatants were collected, centrifuged, stored and used for sequencing the full-length DNA of the genomic fragment 4 of the influenza virus encoding the virus HA (hemagglutinin).

### Cloning and Mutagenesis of HA

Hemagglutinin (HA) of A/PR/8/34 (H1N1) was amplified using the following PCR oligonucleotides:

APR834_s:
(SEQ ID NO: 6)
5′-CACCATGAAGGCAAACCTACTGGTCCTGTTATGTG-3′;
APR834_as:
(SEQ ID NO: 7)
5′-TCAGATGCATATTCTGCACTGCAAAGATCCATTAGA-3′.

The PCR products were cloned into the vector pcDNA 3.1D/V5-His-TOPO (Invitrogen).

Subsequently, mutants for H1N1 hemagglutinin (HA) and H₃N₂ HA were generated by using the Gene Tailor Site-Directed Mutagenesis System (Invitrogen). Twenty HA mutants for H1N1 and 4 HA mutants for H₃N₂ were generated in total.

### FACS Binding Assay

The binding activity of PN-SIA28 was assessed by using wild-type and mutant full-length HA proteins cloned as described above. In brief, human epithelial kidney (HEK) cells 293T were transfected with 4 μg of vector pcDNA 3.1D/V5-His-TOPO containing the HA nucleotide sequences. Following centrifugation and fixation with 4% paraformaldehyde, the transfected cells were incubated for 30 minutes at room temperature with IgG PN-SIA28 (10 μg/ml) or with a murine anti-H1 or anti-H3 (1 μg/ml). The cells were then washed and incubated for 30 minutes at room temperature with human or murine FITC-conjugated monoclonal anti-Fab antibodies. Thereafter, the cells were washed and analyzed by FACS. Non-transfected cells were also included in each experiment as a negative control. A monoclonal anti-murine H1 or H3 subtype antibody directed against a linear epitope was used to assess the transfection efficiency for each HA. The FACS analysis was carried out on cells wherein the signal obtained from those only stained with the secondary antibody was subtracted. The binding of PN-SIA28 to the different mutants was expressed as a binding percentage compared to the wild-type.

### Software

The following software were used for the analysis of the sequences: SeqScape (Applied Biosystems), ClustalX (Toby Gibson), Bio Edit (Tom Hall, Ibis Therapeutics), and Treeview (GubuSoft). RasMol (Roger Sayle), Jmol (Jmol: a Java open-source viewer for 3D chemical structures. http://www.jmol.org/), Cn3D (United States National Library of Medicine, NLM), Pepitope server (Pepitope: mapping of epitopes from affinity-selected peptides. Bioinformatics 2007 23(23):3244-3246.), Mimox (BMC Bioinformatics 2006, 7:451 doi:10.1186/1471-2105-7-451) were used for visualizing and reproducing the molecules. Finally, GraphPad Prism was used for the analysis of the data and the graphical editing.

### Results

### Characterization of the H1N1 Virus Hemagglutinin Epitope Recognized by the Monoclonal Antibody

### Selection and Characterization of Mutants Capable of Escaping the Antibody Response Under Fab PN-SIA28 Selective Pressure

After having subjected the cells to the many steps described in the Methods, the cells infected with the wild type A/PR/8/34 H1N1 virus exhibited a strong cytopathic effect (CPE) both in the absence of antibody PN-SIA28 and in the presence of antibody e509 anti-E2/HCV used as a negative control. Cells infected with the wild type virus showed no evidence of CPE in the presence of PN-SIA28 at a concentration of 10 μg/ml. In contrast, cells infected with the selected variant virus showed a strong CPE in spite of the presence of PN-SIA28 at a concentration of 10 μg/ml. The sequencing of the generated variants capable of escaping the antibody response showed two different mutants, each of which having a single amino acid mutation in the HA2 subunit of the hemagglutinin stem region, compared to the amino acid sequence of the wild type virus. The two mutations are Ile361Thr and Asp362Gly (numbering referred to the linear A/PR/8/34 (H1N1) HA amino acid sequence, NCBI accession number EF467821.1, SEQ ID NO:5 in the sequence listing).

Either Isoleucine (I) or Valine (V) may be naturally present in position 361 in different strains of different subtypes. As described later, the 1361V mutation does not affect the binding of PN-SIA28 to HA. No natural isolates exists which have Thr in position 361 or Gly in position 362.

Identification of the HA Region that Binds mAb PN-SIA28

The antibody PN-SIA28 has a strong neutralizing activity against the influenza A virus. In particular, PN-SIA28 has a heterosubtype neutralizing activity against H1-(such as H1N1) and H3-(such as H₃N₂) subtype viruses, which belong to group 1 and group 2, respectively.

PN-SIA28, when tested in a hemagglutination inhibition assay, did not inhibit the virus-induced clustering of erythrocytes.

The Western blot analysis showed that only the immature form of HA (HAO) is recognized by PN-SIA28.

On the whole, the data obtained from the generation of antibody escape mutants, the hemagglutination inhibition assay and the Western blot analysis, suggest that the region recognized by the neutralizing antibodies does not lie within the HA globular region but in the stem region.

On the basis of these observations, HA molecules containing amino acid substitutions that may prevent the binding of the above-mentioned antibody were generated in order to characterize the epitope recognized by PN-SIA28.

In the following description, the amino acid residues are numbered on the basis of the linear A/PR/8/34 (H1N1) HA sequence, NCBI accession number EF467821.1, SEQ ID NO:5 in the sequence listing.

Firstly, the inventors considered that under the PN-SIA28 selective pressure, two different A/PR/8/34 (H1N1) HA2 antibody escape mutants were generated, in one of which the residue Ile361 was mutated into Thr and in the other the residue Asp362 was mutated into Gly. These two mutants are not recognized by PN-SIA28 anymore. On the basis of these results, HA mutants having an alanine in position 361 (substitution Ile361A1a) or an alanine/glycine in position 362 (substitution Asp362Ala, Asp362Gly) were generated and the ability of PN-SIA28 to bind the mutants was assessed by FACS analysis. PN-SIA28 could bind only weakly to the mutated HAs (Ile361Ala, Asp362Ala, Asp362Gly), demonstrating that these two residues are included in the epitope recognized by this antibody. It is important to note that the amino acid residues Ile361 and Asp362 are highly conserved in many hemagglutinin subtypes (H1, H2, H3, H4, H5, H6, H7, H8, H10, H14, H15).

A second group of mutants with mutations targeted to the possible region recognized by this IgG was designed in order to characterize in detail the epitope of PN-SIA28 HA. Twenty mutants containing a substitution alanine in several amino acid positions, including Ile361Ala and Asp362Ala, were generated in total, see Table 2.

TABLE 2

| HA1 | | HA2 | |
|---|---|---|---|
| I. | His25 (His18 F10 | I. | Trp357 |
| | and CR6261) | II. | Thr358 |
| II. | His45 (His38 F10 | III. | Gly359 |
| | and CR6261) | IV. | Met360 |
| III. | Thr315 (Thr318) | V. | Ile361 (Val18 F10) |
| IV. | Asn336 | VI. | Asp362 (Asp19 F10 and C179) |
| V. | Ile337 | VII. | Gly363 (Gly20 F10 and C179) |
| VI. | Pro338 | VIII. | Trp364 (Ttp21 F10, CR6261, C179) |
| | | IX. | Thr384 (Thr41 F10 and CR6261) |
| | | X. | Ile388 (Ile45 F10 and CR6261) |
| | | XI. | Thr392 (Thr49 F10, CR6261 and C179) |
| | | XII. | Val395 (Val 52 F10, CR6261 and C179) |
| | | XIII. | Asn396 (Asn53 F10 and C179) |
| | | XIV. | Glu400 (Glu57 C179) |

In Table 2, the numbering is based upon the linear A/PR/8/34 (H1N1) HA sequence, NCBI accession number EF467821.1, SEQ ID NO:5 in the sequence listing. The numbering based on the original publications in which the antibodies F10, CR6261 and C179 had been described is provided in brackets.

### Amino Acid Residues of the Hemagglutinin Region Recognized by IgG PN-SIA28

The FACS analysis showed that the binding of the antibody PN-SIA28 was decreased in the following mutants: HA1-I, -II, -III, and -IV; HA2-II, -IV, -V, -VI, -VII, -VIII, -IX, -XI, XII and -XIV (cf. Table 2). These results indicate that the residues His25, His45, Thr315, Asn336, Thr358, Met360, Ile361, Asp362, Gly363, Trp364, Thr384, Thr392, Va1395 and Glu400 are critical for the interaction between PN-SIA28 and HA. Thus, these residues are involved in the binding of the antibody to the stem region of HA. All the other mutations indicated in Table 2 did not result in a decrease in the binding of PN-SIA28 to HA.

An extra HA mutant was generated for position HA2-Ile361, wherein the original residue was changed into Valine. Within the same subtype, the natural sequence may contain in position 361 either an Isoleucine residue or a Valine residue. When the interaction of PN-SIA28 with the mutant HA Ile361Val was tested, no decrease in the interaction was observed. These results indicate that the antibody PN-SIA28 is capable of binding both of the virus variants, both the one with Isoleucine in position 361 and the one with Valine in the same position. This indicates that PN-SIA28 is able to bind and neutralize all the virus strains, irrespective of which of the two residues is present in position 361.

### Differences Between the Epitopes Recognized by IgG PN-SIA28 and by the Antibody C179

The murine monoclonal antibody C179, described in the state of the art, is able to bind a common epitope in the stem region of HA, shared by the HAs of subtypes H1, H2 and H5. The binding of the antibody C179 to this region inhibits the fusion activity of HA and thus results in the neutralization of the virus. The epitope recognized by C179 is composed of two different sites, one located in the HA1 subunit and defined by residues 318-322, and the other located in the HA2 subunit and defined by residues 47-58.

As the two sites are located close together in the center of the stem region of the HA molecule, C179 appears to recognize these sites conformationally.

The mutant viruses capable of escaping the antibody response, containing HAs that carry one Thr to Lys substitution in position 318 of the HA1 subunit or one Val to Glu substitution in position 52 of the HA2 subunit, were not recognized and neutralized by C179 anymore.

The epitope recognized by PN-SIA28 was compared to the one recognized by C179.

The results from the FACS analysis showed that the amino acids His25, His45, Thr315, Thr358, Met360, Ile361, Asp362, Gly363, Trp364, Thr384 and Va1395 (corresponding to mutants HA1-I, -II, and -III; HA2-II, —IV, -V, -VI, -VII, -VIII, -IX, and -XII, cf. Table 2) are important for the binding of PN-SIA28 to HA. These are not described as key residues for the binding of C179 to HA. Moreover, Asn336, Thr392, Va1395 and Glu400 (corresponding to mutants HA1-IV; HA2-XI, -XII and -XIV) are key residues for the interaction of HA with PN-SIA28, as well as for C179. In addition, residue Asn396 (corresponding to the mutant HA2-XIII) is involved in the interaction between C179 and HA, whereas it is not involved in the interaction between PN-SIA28 and HA.

In summary, the epitopes recognized by PN-SIA28 antibody and by C179 antibody are different.

### Differences Between the Epitopes Recognized by IgG PN-SIA28 and by the Antibody CR6261

The antibody CR6261, described in the state of the art, has a heterosubtype neutralizing activity against the H1, H2, H5, H6, H8 and H9 subtypes of the influenza A virus. Crystallographic studies performed on the CR6261-A/South Carolina/1/1918 interaction showed that the antibody recognizes a highly conserved helical region located in the stem, proximal to the membrane on HA1 (residues 18, 38, 40, 42, 292) and HA2 (residues 21, 41, 45, 49, 52, 56). The antibody neutralizes the virus by blocking the conformational rearrangement associated with the fusion of the membrane.

The epitope recognized by PN-SIA28 was compared with the one recognized by CR6261.

Residues Thr315, Thr358, Met360, Ile361, Asp362, Gly363, Trp364 and Thr384 (corresponding to mutants HA1-III; HA2-II, -IV, -V, -VI, -VII, -VIII and -IX, cf. Table 2) are important for the binding of PN-SIA28 to HA but are not described as key residues for the binding of CR6261 to HA. Residues His25, His45 (corresponding to mutants HA1-I and -II, cf. Table 2), Thr392 and Va1395 (corresponding to mutants HA2-XII and -XII, cf. Table 2) are key residues for the interaction of HA with PN-SIA28 and CR6261. Residue Ile388 (corresponding to the HA2-X mutant, cf. Table 2) does not appear to be important for the binding of PN-SIA28 to HA, whereas this residue was shown to be in direct contact with the antibody CR6261.

In summary, the epitopes recognized by PN-SIA28 and CR6261 are different.

### Differences Between the Epitopes Recognized by IgG PN-SIA28 and by Antibody F10

F10, an antibody described in the state of the art, is a high-affinity neutralizing antibody directed against hemagglutinin, extremely effective against highly pathogenic subtypes of the H₅N₁ and H1N1 viruses. This antibody inhibits the fusion process subsequent to attachment by recognizing a highly conserved epitope that is located in the hemagglutinin stem region. The crystal structure of F10 complexed with H5 from the A/Vietnam/1203/04 strain was determined. The epitope recognized by F10 includes a hydrophobic pocket formed by the HA2 fusion peptide flanked by HA1 residues on one side and by the HA2 aA helix on the other side. The HA1 region recognized by F10 includes residues 18 and 38, whereas the HA2 one includes residues 18-21, 41, 45, 49, 52, 53, 56. Studies effected with mutants showed that the residues Va152, Asn53 and Ile56 within the segment in HA2 aA, which performs important interactions with F10, strongly decrease or completely abolish the binding of the antibody.

The epitope recognized by PN-SIA28 was compared with the one recognized by F10.

Residues His25, His45, Ile361, Asp362, Gly363, Trp364, Thr384, Thr392 and Va1395 (corresponding to mutants HA1-I and -II; HA2-V, -VI, -VII, -VIII, -IX, -XI and XII, cf. Table 2), important for the binding of PN-SIA28, are described as key residues for the binding of F10 to HA. Instead, residues Thr315, Asn336, Thr358, Met360 and Glu400 (corresponding to mutants HA1-III, —IV; HA2-II, —IV, and -XIV, Table 2) are key residues for the interaction of PN-SIA28 with HA but not for F10. In addition, Ile388 and Asn396 (corresponding to mutants HA2-X, -XIII, cf. Table 2) do not appear important for the binding of PN-SIA28 to HA, whereas this residue was shown to be in direct contact with F10.

In summary, the epitopes recognized by PN-SIA28 and F10 are different.

### Differences Between IgG and Fab PN-SIA28 in the Binding to HA

The HA original residues Thr315, Asn336, Pro338, Thr392, Glu400, when mutated into alanines, are only faintly recognized by IgG PN-SIA28. Instead, Fab PN-SIA28 is still able to bind strongly to these mutants. The present inventors assume that the differences in the binding characteristics may be caused by the steric hindrance due to the bigger size of the IgGs compared to the Fab fragments.

Characterization of the Epitope of the H₃N₂ virus Hemagglutinin Recognized by Human Monoclonal Antibody PN-SIA28

A second group of HA mutants based upon H₃N₂ HA (A/Aichi/2/68) was created in order to assess if the epitope recognized by PN-SIA28 on H1N1 HA was also shared by H₃N₂ HA, see Table 3.

TABLE 3

| HA1 | HA2 |
|---|---|
| His34 (His25 H1N1) | Ile363 (Ile361 H1N1) |
| Asn54 (His45 H1N1) | Asp364 (Asp362 H1N1) |

The numbering is based upon the linear H₃N₂ HA sequence, NCBI accession number EF614251.1, SEQ ID NO:8 in the sequence listing.

### H₃N₂ HA Amino Acid Residues Recognized by PN-SIA28

The FACS analysis showed that the binding of PN-SIA28 to H₃N₂ HA decreased in mutants HA1-I and -II; HA2-I, -II, cf. Table 3. These results indicate that the residues His34, Asn54, Ile363 and Asp364 are critical for the interaction between PN-SIA28 and HA, thus they are a part of the epitope recognized by the antibody and are located in the stem region of HA.

The study with the mutants performed on H₃N₂ HA confirmed that the epitope recognized by PN-SIA28 is shared by the H1N1 and H₃N₂ hemagglutinins, despite the many amino acid differences found in the linear sequences of the two proteins.

The residues involved in the interaction of the antibody PN-SIA28 with HA are localized in highly conserved regions in most of the influenza A virus subtypes.

### 3. Neutralizing Activity of IgG PN-SIA28 Against Further Influenza a Virus Subtypes

The neutralizing activity found for antibody IgG PN-SIA28 and the region that it recognizes allow for some considerations with regard to the neutralizing activity of this monoclonal also against other influenza virus subtypes. In this connection, it is useful to recall that up to now 16 HA subtypes have been distinguished, only three of which have been able to determine the pandemics documented in man (H1, H2 and H3), whereas other three of them (H5, H9 and H7) have been isolated sporadically from humans, but till now have not been able to cause a pandemic (Fouchier R A, Munster V, Wallensten A, et al. Characterization of a novel influenza A virus hemagglutinin subtype (H16) obtained from black-headed gulls. J Virol 2005; 79(5):2814-22).

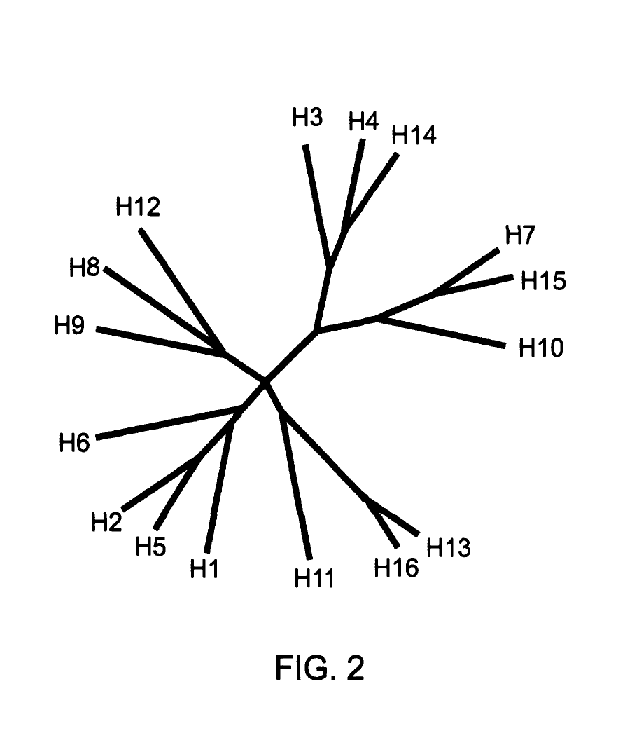

On the basis of the phylogenic distance between the 16 subtypes (from 40% to 60% homology), two phylogenic groups are typically distinguished: group 1 to which H1, H2, H5, H6, H8, H9, H11, H12, H13 and H16 belong, and group 2 to which H3, H4, H7, H10, H14, and H15 belong (Lambert L C, Fauci A S. Influenza vaccines for the future. N Engl J Med 2010; 363(21):2036-44; Nabel G J, Fauci A S. Induction of unnatural immunity: prospects for a broadly protective universal influenza vaccine. Nat Med 2010; 16(12):1389-91). FIG. 2 shows the phylogenic tree of the several hemagglutinin (H) subtypes. Group 1 may be further divided into three clusters, that is cluster H1-like (H1, H2, H5, H6), cluster H11-like (H11, H13 and H16) and cluster H9-like (H9, H8 and H12). Similarly, group 2 may be divided into other two clusters: cluster H3-like (H3, H4 and H14) and cluster H7-like (H7, H10 and H15).

On the basis of the broad neutralizing activity (also extended to the tested H3 isolates) demonstrated by IgG PN-SIA-28 and of the mutual phylogenic distances between the different subtypes, a neutralizing activity thereof is also likely towards isolates belonging to other subtypes. In particular, we can infer that:
1) The neutralizing activity is extremely likely to be extended to isolates belonging to all the cluster H1-like subtypes, that is H2, H5 and H6.
2) The neutralizing activity is very likely to be extended to isolates belonging to the cluster H9-like, that is H9, H8 or H12.
3) The neutralizing activity is likely to be extended to isolates belonging to the cluster H11-like subtypes, that is H11, H13 or H12.
4) The activity towards subtypes belonging to group 2 is less easily predictable. In this connection, we can state that:
   a. The activity of PN-SIA28 is extremely likely to be extended to other isolates belonging to H3 subtype in addition to those already considered.
   b. It is possible that the activity of PN-SIA28 is extended to isolates belonging to other cluster H3-like subtypes, that is H4 and H14.
   c. The activity of PN-SIA28 is less likely to be extended to isolates belonging to cluster H7-like subtypes, that is H7, H10 and H15.

Preliminary experiments were performed to verify these hypotheses, in order to assess the ability of IgG PN-SIA28 to bind recombinant proteins belonging to the subtypes hitherto isolated in the human species. Taking into consideration that the region recognized by PN-SIA28 is a highly neutralizing epitope, this finding allows to associate the ability of binding a certain subtype with the actual ability of neutralizing it.

In this context, it has been decided to proceed by applying an experimental approach already previously used by the present inventors, which comprises the synthesis of artificial genes encoding full-length hemagglutinins belonging to different subtypes, and the subsequent expression thereof on the surface of cells transfected with the individual constructs (Burioni R, Canducci F, Mancini N, et al. Monoclonal antibodies isolated from human B cells neutralize a broad range of H1 subtype influenza A viruses including swine-origin Influenza virus (S-OIV). Virology 2010; 399(1):144-52; Burioni R, Canducci F, Mancini N, et al. Molecular cloning of the first human monoclonal antibodies neutralizing with high potency swine-origin influenza A pandemic virus (S-OIV). New Microbiol 2009; 32(4):319-24). Thus, sequences of genes encoding HA from isolates belonging to subtypes that, in addition to H1 and H3, have already affected humans were selected from online databases (http://www.ncbi.nlm.nih.gov/nuccore; http://openflu.vital-it.ch/browse.php#results).

The following are the selected isolates:
A/Ann Arbor/6/60 (H2N2)
A/Vietnani/1203/2004 Glade 1 (H5N1)
LAIV A/chicken/Hong Kong/G9/97 (H9N2)
A/New York/107/2003 (H7N2)

The transfected cells were then analyzed by immunofluorescence and FACS with PN-SIA28, and the results are summarized in Table 4. The speculations done on the basis of the phylogenic distances between the different subtypes were fully confirmed by the results obtained. Thus, this allows to believe that PN-SIA28's activity is at least extended to subtypes H₂N₂, H₅N₁ and H₉N₂.

TABLE 4
Binding ability of PN-SIA28 towards cells transfected with
hemagglutinins belonging to different subtypes.

| Phylogenic | HA | | | |
|---|---|---|---|---|
| cluster | Subtype | Isolate | IF | FACS |
| H1-like | H2 | A/Ann Arbor/6/60/(H2N2) | + | + |
| | H5 | A/Vietnam/1203/2004 clade 1 | + | + |
| | | (H5N1) | | |
| H9-like | H9 | A/chicken/Hong Kong/G9/97 | + | + |
| | | (H9N2) | | |
| H7-like | H7 | A/New York/107/2003 (H7N2) | − | − |

1-17. (canceled)

18. A human monoclonal antibody against the influenza A virus hemagglutinin antigen comprising a full-length IgG wherein said antibody recognizes a conserved epitope located in the hemagglutinin stem region, said epitope comprising the amino acid residues His 25, His45, Thr315 and Asn336 of the HA1 hemagglutinin subunit and the amino acid residues Thr358, Met360, Ile361 or Va1361, Asp362, Gly363, Trp364, Yhr384, Thr392, Va1395 and Glu400 of the HA2 hemagglutinin subunit, the numbering of said amino acid residues being based on the H1N1 hemagglutinin amino acid sequence designated as SEQ ID NO: 5 and wherein said antibody is capable of binding and neutralizing a plurality of influenza A virus subtypes including at least the H1 subtype and the H3 subtype.

19. The antibody of claim 18, wherein said antibody is capable of binding and neutralizing the H5 subtype.

20. The antibody of claim 18, wherein said antibody is capable of binding and neutralizing the H2 subtype.

21. The antibody of claim 18, wherein said antibody is capable of binding and neutralizing the H9 subtype.

22. The antibody of claim 18, wherein said antibody is capable of binding and neutralizing the H5 subtype and the H2 subtype.

23. The antibody of claim 18, wherein said antibody is capable of binding and neutralizing the H5 subtype and the H9 subtype.

24. The antibody of claim 18, wherein said antibody is capable of binding and neutralizing the H2 subtype and the H9 subtype.

25. The antibody of claim 18, wherein said antibody is capable of binding and neutralizing the H5 subtype, the H2 subtype and the H9 subtype.

26. The antibody of claim 18, comprising a heavy chain variable region which comprises the amino acid sequence SEQ ID NO:1 and a light chain variable region which comprises the amino acid sequence SEQ ID NO:2.

27. The antibody of claim 18, comprising a heavy chain variable region encoded by the nucleic acid sequence SEQ ID NO:3 and a light chain variable region encoded by the nucleic acid sequence SEQ ID NO:4.

28. A pharmaceutical composition comprising the antibody of claim 18 and pharmaceutically acceptable excipients.

29. A method for treating a condition associated with influenza A comprising administering the antibody of claim 18 to a subject in need thereof.

30. The method of claim 29, wherein the condition associated with influenza A virus infection is influenza syndrome.

31. A method for detecting, in a biological sample of a subject, the presence of anti-influenza virus antibodies having heterosubtype cross-neutralizing activity, comprising contacting said biological sample with the antibody of claim 18.

32. A diagnostic kit comprising as a specific reagent the antibody of claim 18 and instructions for use to detect, in a biological sample from a patient, anti-influenza A virus antibodies having heterosubtype cross-neutralizing activity.

33. A method for detecting, in an immunogenic or vaccine composition, the presence of influenza A virus epitopes capable of eliciting, in a subject to which it is administered, anti-influenza A virus antibodies having heterosubtype cross-neutralizing activity towards the influenza A virus, comprising contacting said composition with the antibody of claim 18.

34. A mimotope directed against the idiotype of the antibody of claim 18.


SEQUENCE LISTING (8 sequences)

SEQ ID NO: 1 - protein, 122 aa, Homo sapiens
LEESGGGVVQPGRSLRLSCAASGFPFSSYGMHWVRQAPGKGLEWVAGVSYDGSYKYYADSVKGRFTISRDSSKSTLYLQMNSLRPEDTAVYYCARPSAIFGIYIILNGLDVWGQGTTVTVSS

SEQ ID NO: 2 - protein, 105 aa, Homo sapiens
ELTQSPSSVSASVGDRVTITCRATQGISSWLAWYQQKPGKPPKLLIFGASSLQSGVPSRFSGSGSGTDFTLTISSLQPEDFATYFCQQAHSFPLTFGGGTKVEIK

SEQ ID NO: 3 - DNA, 366 bp, Homo sapiens
ctcgaggagtctgggggaggcgtggtccagcctgggaggtccctgagactctcctgtgcagcctctggattccccttcagtagttatggcatgcactgggtccgccaggctccaggcaaggggctggagtgggtggcaggtgtttcatatgatggaagttataaatactatgcggactccgtcaagggccgattcaccatctccagagacagttccaagagcactctatatctgcaaatgaacagcctgagacctgaggacacggctgtgtattactgtgcgagaccttccgcgatttttggaatatacattattctaaacggtttggacgtctggggccaagggaccacggtcaccgtctcttca

SEQ ID NO: 4 - DNA, 315 bp, Homo sapiens
gagctcacgcagtctccatcttccgtgtctgcatctgtaggagacagagtcactatcacttgtcgggcgactcagggtattagtagttggttagcctggtatcagcagaaaccagggaaaccacctaaactcctgatttttggtgcatctagtttgcaaagtggggtcccatcaaggttcagcggcagtggatctgggacagatttcactctcaccatcagcagtctacagcctgaagattttgcaacttacttttgtcaacaggctcacagtttcccgctcactttcggcggcgggaccaaggtggagatcaaa

SEQ ID NO: 5 - protein, 565 aa, Influenza virus A (H1N1)
MKANLLVLLCALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNNPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 6 - DNA, 35 bp, artificial
  PCR primer
caccatgaaggcaaacctactggtcctgttatgtg

SEQ ID NO: 7 - DNA, 36 bp, artificial
  PCR primer
tcagatgcatattctgcactgcaaagatccattaga

SEQ ID NO: 8 - protein, 566 aa, Influenza A virus (H3N2)
MKTIIALSYIFCLALGQDLPGNDNSTATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGVHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKQTRGLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIESIRNGTYDHDVYRDEALNNRFQIKGVELKSGYKDWILWISFAISCFLLCVVLLGFIMWACQRGNIRCNICI